(19) United States
(12) Patent Application Publication
(10) Pub. No.: US 2024/0041829 A1
(43) Pub. Date: Feb. 8, 2024

(54) METHODS FOR MODULATING MICROBIAL POPULATIONS

(71) Applicants: B.G. NEGEV TECHNOLOGIES AND APPLICATIONS LTD., AT BEN-GURION UNIVERSITY, Be'er Sheva (IL); BAR-ILAN UNIVERSITY, Ramat Gan (IL)

(72) Inventors: Raz JELINEK, Reut (IL); Orit MALKA, Beer Sheva (IL); Omry KOREN, Yesud Hama'ala (IL)

(21) Appl. No.: 18/258,560

(22) PCT Filed: Dec. 23, 2021
(86) PCT No.: PCT/IL2021/051527

§ 371 Date: Jun. 21, 2023

Related U.S. Application Data
(60) Provisional application No. 63/130,464, filed on Dec. 24, 2020.

(51) Int. Cl. A61K 31/404; A61K 35/741; A61K 35/747; A61K 35/20; A61P 29/00; A61K 36/062; A61K 31/05; A61K 35/744; A61P 1/14; A61K 35/00

(52) U.S. Cl. CPC A61K 31/404; A61K 35/741; A61K 35/747; A61K 35/20; A61P 29/00; A61K 36/062; A61K 31/05; A61K 35/744; A61P 1/14; A61K 2035/115

(57) ABSTRACT

The present invention is directed to compositions comprising a Tryptophol derivative, a 4-Ethyl-Phenol derivative, or a combination thereof, and methods of using same, such as for modulating abundance, diversity, or both, of a microbial population tin a subject in need thereof.

### FIELD OF THE INVENTION

The present invention, in some embodiments thereof, is in the field of microbiology.

### BACKGROUND

Probiotic milk-fermented microorganism mixtures (e.g., yogurt, kefir) are perceived as contributing to human health, and possibly capable of protecting against bacterial infections. Co-existence of probiotic microbiomes are likely maintained via complex biomolecular mechanisms, secreted metabolites mediating cell-cell communication, and other yet-unknown biochemical pathways. In particular, deciphering molecular mechanisms by which probiotic microorganisms inhibit proliferation of pathogenic bacteria would be highly important for understanding both the potential benefits of probiotic foods as well as maintenance of healthy gut microbiome.

There is still a great need for methods for modulating microbial populations, e.g., a host microbiome, such that the abundance and/or diversity of beneficiary microorganisms is increased.

### SUMMARY

The present invention, in some embodiments, is directed to a method for modulating a microbial population of a host.

The present invention is based, in part, on the surprising findings that a combination of a Tryptophol derivative and a 4-Ethyl-Phenol derivative have successfully increased the abundance of the bacterial family of Odoribacteraceae, in two separate inflammation modalities. It is known that the abundance, diversity, or both, of this bacterial family is reduced in cases of inflammation, obesity, and other deficiencies (e.g., Vitamin D deficiency).

Therefore, the present invention can be utilized to increase the abundance, diversity, or both, of beneficiary bacteria (e.g., Odoribacteraceae) in a subject in need of such treatment, such as, but not limited to, in cases of inflammatory diseases or conditions.

According to a first aspect, there is provided a composition comprising a Tryptophol derivative, a 4-Ethyl-Phenol derivative, or a combination thereof, and an acceptable carrier, for use in the treatment of a subject in need of microbial population modulation.

According to another aspect, there is provided a method for modulating abundance, diversity, or both, of a microbial population in a subject in need thereof, comprising administering to the subject a therapeutically effective amount of a composition comprising a Tryptophol derivative, a 4-Ethyl-Phenol derivative, or a combination thereof, thereby modulating abundance, diversity, or both, of a microbial population in the subject.

In some embodiments, modulating comprises increasing or decreasing.

In some embodiments, increasing or decreasing is by at least 5% compared to a control.

In some embodiments, modulating comprises increasing the abundance, diversity, or both, of bacteria belonging to the phylum of Bacteriodetes.

In some embodiments, modulating comprises increasing the abundance, diversity, or both, of bacteria belonging to the family of Odoribacteraceae.

In some embodiments, modulating comprises reducing the abundance, diversity, or both, of bacteria belonging to a family selected from the group consisting of Vibrionaceae, Enterobacteriaceae, Staphylococcaceae, Pseudomonadaceae, Helicobacteraceae, and any combination thereof.

In some embodiments, modulating comprises reducing the abundance, diversity, or both, of bacteria selected from the group consisting of Vibrio cholerae, salmonella enterica, staphylococcus aureus, Pseudomonas aeruginosa, Helicobacter pylori, and any combination thereof.

In some embodiments, the composition comprises any one of the Tryptophol derivative and the 4-Ethyl-Phenol derivative in a concentration ranging from 1 μM to 100 μM.

In some embodiments, the Tryptophol derivative is Tryptophol acetate. In some embodiments, the 4-Ethyl-Phenol derivative is Tyrosol acetate.

In some embodiments, the microbial population comprises a skin microbial population, a gut microbial population, a vaginal microbial population, or any combination thereof, of said subject.

In some embodiments, modulating abundance, diversity, or both, of the microbial population in the subject comprises modulating abundance, diversity, or both, of the microbial population in skin, digestive system, oral cavity, respiratory system, a body topical surface, gut, vagina, or any combination thereof, of the subject.

In some embodiments, the subject is afflicted with an inflammatory disease.

In some embodiments, the subject is afflicted with an inflammatory bowel disease (IBD).

In some embodiments, IBD comprises any one of Crohn's disease and ulcerative colitis.

In some embodiments, the composition is a pharmaceutical composition or a nutraceutical composition.

In some embodiments, the composition further comprises a microorganism mixture.

In some embodiments, the microorganism mixture comprises Kluyveromyces marxianus and at least one probiotic microorganism, and wherein the microorganism mixture comprises at least 3% K. marxianus.

In some embodiments, the at least one probiotic microorganism is a probiotic bacterium.

In some embodiments, the probiotic bacterium is selected form the group consisting of Lactobacillus, Propionibacterium, Lactococcus, and Leuconostoc.

In some embodiments, the microorganism mixture is suspended in a medium.

In some embodiments, the medium is milk.

In some embodiments, the microorganism mixture is kefir.

In some embodiments, the Tryptophol derivative and the 4-Ethyl-Phenol derivative are produced by K. marxianus.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIGS. 1A-1C include graphs and a table showing comparative microbiome sequencing of mice post treatment with dextran sulphate sodium (DSS) and +Molecules (50 μM or 75 μM). (1A) Alpha diversity metrics from the baseline microbiome samples output. Shannon's diversity index (p=0.028). (1B) Beta diversity metrics showing the difference between microbial communities (difference in taxonomic abundance profiles) weighted UniFrac (p=0.029). (1C) The bacterial composition was analyzed at class, order, family, genus and species levels. Differential abundance between groups at each taxonomic level was tested using ANOVA-like Differential Gene Expression analysis (ALDEx2).

FIGS. 2A-2C include graphs showing the effect of tryptophol acetate and tyrosol acetate mixture treatment on mice microbiome. (2A and 2B) Principal coordinate analysis plot representing beta-diversity based on weighted UniFrac distances after (2A) 6 hours (permutational multivariate analysis of variance, P=0.001) and (2B) 24 hours of treatment (permutational multivariate analysis of variance, P=0.038). (2C) Bacterial taxa Bacteroides ovatus relative abundance (%) boxplots for untreated and molecules treated groups. The taxa were more abundant in treated group 24 h from LPS injection. (Treated and untreated, n=8)

### DETAILED DESCRIPTION

The present invention, in some embodiments thereof, relates to a method for modulating or altering a microbial population of a host. In some embodiments, modulating comprises increasing or decreasing the abundance, diversity, or both, of a microbial population of a host.

The terms “modulating”, “altering”, and “modifying” are used herein interchangeably and comprise increasing or decreasing.

As used herein, the term “compound or molecule of the invention” refers to any Tryptophol derivative or 4-Ethyl-Phenol derivative, as described herein below, having any one of antimicrobial activity, anti-inflammatory activity, and anti-amyloid aggregation activity.

As used herein, the term “microbial population” refers to a combination comprising at least 2 sub-populations of microorganisms, e.g., of different species, genus, phylum, etc. In some embodiments, the microbial population comprises any one of bacteria, virus, fungus, protozoan, and any combination thereof. In some embodiments, a microbial population comprises a microbiota or a microbiome.

As used herein, the terms “microbiota” and “microbiome” are interchangeable and refer to a combination of living bacteria and other microorganisms that live in or on a host, such as, but not limited to a human host.

### Methods of Use

According to some embodiments, there is provided a method for modulating abundance, diversity, or both, of a microbial population in a subject in need thereof, comprising administering to the subject a therapeutically effective amount of a composition comprising a Tryptophol derivative, a 4-Ethyl-Phenol derivative, or a combination thereof, thereby modulating abundance, diversity, or both, of a microbial population in the subject.

In some embodiments, the method is for treating or preventing a disease in a subject in need thereof.

In some embodiments, modulating comprises or is increasing. In some embodiments, modulating comprises or is enhancing. In some embodiments, modulating comprises or is or decreasing. In some embodiments, modulating comprises or is or lowering.

In some embodiments, the method comprises modulating abundance, diversity, or both, of a microbial population in skin, digestive system, oral cavity, respiratory system, a body topical surface, gut, or any combination thereof, of the subject.

In some embodiments, microbial population targeted for modulation or modulated according to the herein disclosed method reside in or on the skin, digestive system, oral cavity, respiratory system, a body topical surface, gut, vagina (including vaginal environment) or any combination thereof, of the subject.

Methods for determining the abundance, diversity, or both, of microbial population are common and would be apparent to one of ordinary skill in the art. Non-limiting examples of methods for determining the abundance, diversity, or both, of microbial population include, but are not limited to, sequencing and/or next generation sequencing, and subsequent Shannon's diversity index, and beta diversity indices analyses, such as exemplified herein. Other analysis may include operational taxonomic units (OTUs) analysis wherein and each OTU is aligned with known nucleotide sequences of available databases (e.g., 16S rDNA sequence databases Silva, Greengenes, Ribosomal Database Project or chaperonin database cpnDB). The sequences are then assigned according to the current taxonomy and analyzed phylogenetically. The taxa resulting from the analyzes are represented according to their relative frequency in the analyzed sequence data set.

In some embodiments, increasing or increase comprises at least 5%, at least 15%, at least 25%, at least 40%, at least 50%, at least 75%, at least 100%, at least 250%, at least 350%, at least 500%, at least 750%, at least 850%, or at least 1,000% increase, or any value and range therebetween. Each possibility represents a separate embodiment of the invention. In some embodiments, increasing or increase comprises 5 to 1,000% increase.

In some embodiments, decreasing or decrease comprises at least 5%, at least 15%, at least 25%, at least 30%, at least 40%, at least 50%, at least 60%, at least 70%, at least 80%, at least 90%, at least 95%, at least 99%, or at least 100% decrease, or any value and range therebetween. Each possibility represents a separate embodiment of the invention. In some embodiments, decreasing or decrease comprises 5 to 100% decrease.

In some embodiments, increasing or decreasing is compared to a control. In some embodiments, a control comprises a healthy subject. In some embodiments, a control comprises a sample derived or obtained from a healthy subject. In some embodiments, a sample comprises a biological sample. Obtaining a biological sample is well within the capabilities of a skilled artisan. In some embodiments, a control comprises a sample derived or obtained from a subject not afflicted with an inflammatory disease or a condition associated therewith. In some embodiments, a control comprises a sample derived or obtained from a subject not afflicted with inflammation.

In some embodiments, the method comprises increasing the abundance, diversity, or both, of bacteria belonging to the phylum of Bacteriodetes.

In some embodiments, the method comprises increasing the abundance, diversity, or both, of bacteria belonging to the family of Odoribacteraceae.

In some embodiments, the method comprises reducing the abundance, diversity, or both, of bacteria belonging to a family selected from Vibrionaceae, Enterobacteriaceae, Staphylococcaceae, Pseudomonadaceae, Helicobacteraceae, or any combination thereof.

In some embodiments, the method comprises reducing the abundance, diversity, or both, of bacteria selected from Vibrio cholerae, salmonella enterica, staphylococcus aureus, Pseudomonas aeruginosa, Helicobacter pylori, or any combination thereof.

In some embodiments, the microbial population comprises a skin microbial population. In some embodiments, the microbial population comprises a gut microbial population. In some embodiments, the microbial population comprises a skin microbial population and a gut microbial population. In some embodiments, the microbial population is a microbial population of a subject (e.g., a host).

In some embodiments, the subject is afflicted with an inflammatory disease.

In some embodiments, the subject is afflicted with an infectious disease.

In some embodiments, the subject is afflicted with an inflammatory bowel disease (IBD).

In some embodiments, IBD comprises any one of Crohn's disease and ulcerative colitis. In one embodiment, IBD is Crohn's disease. In one embodiment, IBD is ulcerative colitis.

Non-limiting examples of infectious diseases include urinary tract infection, gastrointestinal infection, enteritis, salmonellosis, diarrhea, nontuberculous mycobacterial infections, legionnaires' disease, hospital-acquired pneumonia, skin infection, cholera, septic shock, periodontitis, infection, inflammatory bowel disease, ulcerative colitis (UC), Crohn's disease, and sinusitis. In some embodiments, the infection induces a condition selected from the group consisting of bacteremia, skin infections, neonatal infections, pneumonia, endocarditis, osteomyelitis, toxic shock syndrome, scalded skin syndrome, and food poisoning.

The term “subject” as used herein refers to an animal, more particularly to non-human mammals and human organism. Non-human animal subjects may also include prenatal forms of animals, such as, e.g., embryos or fetuses. Non-—limiting examples of non-human animals include horse, cow, camel, goat, sheep, dog, cat, non-human primate, mouse, rat, rabbit, hamster, guinea pig, and pig. In one embodiment, the subject is a human. Human subjects may also include fetuses.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described peptides prior to the induction or onset of the disease/disorder process. This could be done where an individual has a genetic pedigree indicating a predisposition toward occurrence of the disease/disorder to be prevented. For example, this might be true for an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

As used herein, the term “condition” includes anatomic and physiological deviations from the normal that constitute an impairment of the normal state of the living animal or one of its parts, that interrupts or modifies the performance of the bodily functions.

### Tryptophol Derivative

In some embodiments, a Tryptophol derivative of the invention has the structure

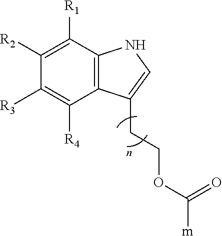

wherein “R1 ”, “R2”, “R3”, and “R4” are selected from the group consisting of methyl (CH3), ethyl (CH3CH2), propyl (CH3CH2CH2) and butyl (CH3CH2CH2CH2);“n” is a carbon chain comprising one, two, three or four carbons; and “m” is selected from the group consisting of methyl (CH3), ethyl (CH3CH2), propyl (CH3CH2CH2) and butyl (CH3CH2CH2CH2).

In some embodiments, the Tryptophol derivative is Tryptophol acetate.

Tryptophol acetate is known in the art as having the structure

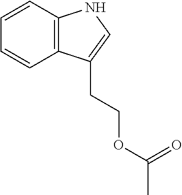

### 4-Ethyl-Phenol Derivative

In some embodiments, a 4-Ethyl-Phenol derivative has the structure

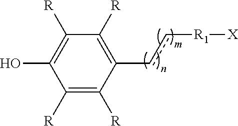

wherein each R is independently selected from the group consisting of hydroxyl, hydrogen, methyl (CH3), ethyl (CH3CH2), propyl (CH3CH2CH2) and butyl (CH3CH2CH2CH2); “n”, “m” are from 1 to 4, R1 comprises a heteroatom or is absent [symbol] represents a bond selected from the group consisting of sp3 single C—C bond, sp2 double C—C bond, sp triple C—C bond; and X is selected from the group consisting of a carboxylic acid derivative, an alkyl, and hydrogen.

In some embodiments, a 4-Ethyl-Phenol derivative has the structure

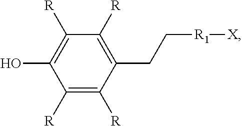

wherein R, R1 and X are as described hereinabove.

In some embodiments, each R is independently selected from hydroxyl, and hydrogen.

In some embodiments, R1 is selected from O, NH, and NH2.

In some embodiments, 4-Ethyl-Phenol derivative is a dopamine derivative represented by formula

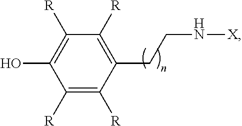

or by formula

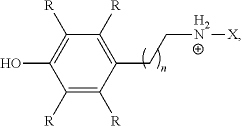

wherein R and X are as described hereinabove.

In some embodiments, a dopamine derivative is represented by formula

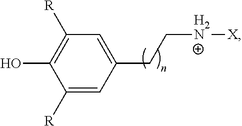

or by formula

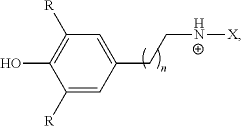

wherein R and X are as described hereinabove.

wherein R and X

In some embodiments, X is hydrogen.

In some embodiments, 4-Ethyl-Phenol derivative is dopamine or a salt thereof.

In some embodiments, a 4-Ethyl-Phenol derivative has the structure

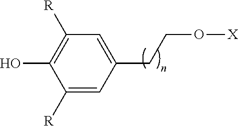

wherein each R is independently selected from hydroxyl, and hydrogen; and X is selected from the group consisting of a carboxylic acid derivative, an alkyl, and hydrogen.

In some embodiments, X is

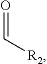

wherein R2 is selected from —OH, —SH, —NH2, thioalkyl, oxyalkyl, aminoalkyl, hydrogen, alkyl, substituted alkyl.

In some embodiments, R2 is hydrogen or an alkyl.

In some embodiments, R2 is a C1 -C5 alkyl.

In some embodiments, R2 is hydrogen.

In some embodiments, the 4-Ethyl-Phenol is a derivative of Tyrosol acetate.

Tyrosol acetate is known in the art as having the structure

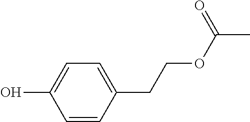

In some embodiments, a 4-Ethyl-Phenol derivative is a derivative of caffeic acid having the structure

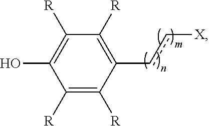

wherein each R is independently selected from hydroxyl, hydrogen, methyl (CH3), ethyl (CH3CH2), propyl (CH3CH2CH2) and butyl (CH3CH2CH2CH2); “n”, “m” are from 1 to 4, [symbol] represents a bond selected from the group consisting of sp3 single C—C bond, sp2 double C—C bond, sp triple C—C bond; and X is selected from a carboxylic acid derivative, an alkyl, and hydrogen.

In some embodiments, each R is independently selected from hydroxyl, and hydrogen.

In some embodiments, [symbol] represents an unsaturated C—C bond. In some embodiments, [symbol] represents a double C—C bond.

In some embodiments, X is selected from a carboxylic acid derivative, and hydrogen.

In some embodiments, the derivative of caffeic acid has the structure

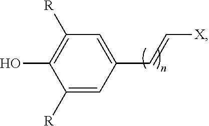

wherein R and X are as defined hereinabove.

In some embodiments, the derivative of caffeic acid has the structure

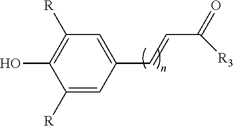

wherein R is as defined hereinabove, and R₃ is selected from hydrogen, —OH, —SH, —NH₂, thioalkyl, oxyalkyl, aminoalkyl, hydrogen, alkyl, substituted alkyl.

In some embodiments, R is selected from hydrogen, —OH, and alkyl.

In some embodiments, the derivative of caffeic acid has the structure

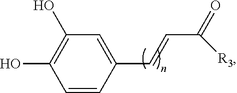

wherein R3 is as defined hereinabove.

As used herein, the term “carboxylic acid derivative” encompasses carboxy, amide, carbonyl, anhydride, carbonate ester, and carbamate.

As used herein, the term “derivative” encompasses any compound having antimicrobial activity that is generated from a similar compound by a chemical reaction, or any compound produced from another compound by substitution of one or more atoms. In some embodiments, the derivative comprises a structural analog.

In some embodiments, a derivative as disclosed herein is obtained by any chemical modification of Tryptophol or 4-Ethyl-Phenol, as long as it has the ability to modulate abundance, diversity, or both, of a microbial population. In some embodiments, Tryptophol or 4-Ethyl-Phenol are chemically modified by adding at least one chemical group selected from acetylation, methylation, phosphorylation, amidation or others. In some embodiments, a chemical modification comprises substitution. In some embodiments, the modification comprises the addition of an acetate group to Tryptophol or 4-Ethyl-Phenol. In some embodiments, a Tryptophol acetate or Tyrosol acetate further comprises at least one chemical group as described above.

As used herein, a Tryptophol derivative does not comprise Tryptophol.

As used herein, a 4-Ethyl-Phenol derivative does not comprise Tyrosol.

In some embodiments, the disclosed invention is directed to a composition comprising at least one molecule selected from a Tryptophol derivative, a 4-Ethyl-Phenol derivative, and any combination thereof, and at least one pharmaceutically acceptable carrier or diluent.

In some embodiments, the composition comprises Tryptophol acetate, Tyrosol acetate, or any combination thereof, and at least one pharmaceutically acceptable carrier or diluent.

In some embodiments, the Tryptophol derivative and/or the 4-Ethyl-Phenol derivative is chemically synthesized or biosynthesized. Methods of biosynthesis are well known within the art, and can include, but are not limited to production in a cell culture or enzymatic cell-free production. In some embodiments, Tryptophol derivative and/or the 4-Ethyl-Phenol derivative is biosynthesized using a cell culture comprising Kluyveromyces marxianus. In some embodiments, a culture comprising K. marxianus is a mono- or poly-culture. In some embodiments, the Tryptophol derivative and/or the 4-Ethyl-Phenol derivative is biosynthesized by K. marxianus. In some embodiments, the Tryptophol derivative and/or the 4-Ethyl-Phenol derivative are biosynthesized by K. marxianus according to the method of the present invention. In some embodiments, Tryptophol acetate or Tyrosol acetate are biosynthesized by K. marxianus according to the method of the present invention.

According to some embodiments, there is provided a composition comprising a Tryptophol derivative, a 4-Ethyl-Phenol derivative, or a combination thereof, and an acceptable carrier.

In some embodiments, the composition is suitable for use in the treatment of a subject in need of microbial population modulation.

In some embodiments, the composition is for use in the treatment of a subject in need of microbial population modulation.

In some embodiments, the composition comprises any one of a Tryptophol derivative and a 4-Ethyl-Phenol derivative in a concentration ranging from 1 μM to 100 μM, 10 μM to 100 μM, 15 μM to 80 μM, 25 μM to 75 μM, 50 μM to 90 μM, or 50 μM to 75 μM. Each possibility represents a separate embodiment of the invention.

In some embodiments, the composition comprises Tryptophol acetate.

In some embodiments, the composition comprises Tyrosol acetate, dopamine HCl, caffeic acid, or any combination thereof. In some embodiments, the composition comprises Tyrosol acetate.

In some embodiments, the composition comprises a combination of Tryptophol acetate and any one of Tyrosol acetate, dopamine HCl, caffeic acid, or any combination thereof. In some embodiments, the composition comprises a combination of Tryptophol acetate and Tyrosol acetate.

In some embodiments, the composition further comprises a microorganism mixture.

In some embodiments, the microorganism mixture comprises Kluyveromyces marxianus and at least one probiotic microorganism. In some embodiments, the microorganism mixture comprises at least 3% K. marxianus.

In some embodiments, the at least one probiotic microorganism is a probiotic bacterium.

In some embodiments, the probiotic bacterium is selected form the group consisting of Lactobacillus, Propionibacterium, Lactococcus, and Leuconostoc.

In some embodiments, the microorganism mixture is suspended in a medium.

In some embodiments, the medium is milk.

In some embodiments, the microorganism mixture is or comprises kefir.

In some embodiments, the Tryptophol derivative, the 4-Ethyl-Phenol derivative, or both, are produced by K. marxianus.

In one embodiment, K. marxianus is K. marxianus strain HA 63 [NRRL Y-8281, CBS 712].

As used herein, the term “probiotic” refers to any substance and/or a microorganism that promotes growth, especially of microorganisms with beneficial properties (e.g., intestinal flora).

In some embodiments, a microorganism content (%) within the microorganism mixture is quantified according to the portion of the microorganism's DNA out of the total DNA of the mixture. In some embodiments, DNA quantification is based directly on the amount of DNA extracted. In some embodiments, DNA quantification further comprises enzymatic reaction, including but not limited to restriction, ligation, amplification, sequencing, or any combination thereof. In some embodiments, DNA quantification is based on next generation sequencing. In some embodiments, DNA quantification is based on the ratio of the amount of microorganism-specific DNA reads compared to the total number of DNA reads of the microorganism mixture.

In some embodiments, a microorganism content within the microorganism mixture comprises the number of cells of the microorganism per volume of the microorganism mixture culture (e.g., colony forming unit [CFU]). In some embodiments, a microorganism content within the microorganism mixture comprises the number of cells of the microorganism compared to the total number of cells in the microorganism mixture. In some embodiments, a microorganism content within the microorganism mixture comprises the CFU of the microorganism.

In some embodiments, at least 1%, at least 3%, at least 5%, at least 7%, at least 10%, at least 15%, at least 20%, at least 30%, at least 35%, at least 40%, at least 45%, at least 50%, at least 55%, at least 60%, at least 65%, or at least 70% of cells in the microorganism mixture are K. marxianus cells, or any value and range therebetween. Each possibility represents a separate embodiment of the invention. In some embodiments, 1-4%, 2-5%, 4-7%, 6-11%, 10-16%, 15-22%, 20-32%, 30-35%, 32-40%, 38-48%, 45-55%, or 55-75%, 60-80%, 65-90%, or 80-100% of cells in the microorganism mixture are K. marxianus cells. Each possibility represents a separate embodiment of the invention.

In some embodiments, the composition comprises at least one probiotic bacterium. In one embodiment, a probiotic bacterium is selected from the genus Lactobacillus. In one embodiment, a probiotic bacterium is selected from the genus Propionibacterium. In one embodiment, a probiotic bacterium is selected from the genus Lactococcus. In one embodiment, a probiotic bacterium is selected from the genus Leuconostoc. In some embodiments, at least one probiotic bacterium comprises at least two, at least three, at least four, at least five, at least six, at least seven, at least eight, at least nine, or at least ten probiotic bacteria, or any value and range therebetween. Each possibility represents a separate embodiment of the invention. In some embodiments, 30% at most, 25% at most, 20% at most, 15% at most, 10% at most, 5% at most, or 1% at most, of cells in the microorganism mixture are probiotic bacteria, or any value and range therebetween. Each possibility represents a separate embodiment of the invention. In some embodiments, 1-5%, 4-10%, 8-18%, 12-20%, 17-25%, or 22-30% of cells in the microorganism mixture are probiotic bacteria. Each possibility represents a separate embodiment of the invention.

In some embodiments, the microorganism mixture further comprises other types of microorganisms. In one embodiment, the other microorganisms are not probiotic microorganisms, such as yeast or bacteria. In some embodiments, 15% at most, 13% at most, 11% at most, 10% at most, 9% at most, 7% at most, 5% at most, 4% at most, 3% at most, 2% at most, or 1% at most of cells in the microorganism mixture belong to a microorganism type other than a probiotic yeast and at least one probiotic bacteria, or any value and range therebetween. Each possibility represents a separate embodiment of the invention. In some embodiments, the microorganism mixture comprises no other type of microorganism except probiotic yeast and at least one probiotic bacteria.

In some embodiments, the microorganism mixture is suspended in a medium. In some embodiments, the microorganism mixture is grown in the medium. In some embodiments, the medium is a cell culture medium suitable for growth and maintenance of the microorganism mixture. In one embodiment, the cell culture medium is optimized for microorganism growth, such as, but not limited to, milk.

As used herein, “cell culture medium” refers to any medium, liquid, semi solid, or solid, which enables cells proliferation. Cell culture media are known in the art and can be selected, depending on the type of cell to be grown. For example, a cell culture medium for use in growing cells is Luria-Bertani broth (LB; Miller's broth). In some embodiments, microorganism mixture is cultured under effective conditions, which allow for increased yield of production from the culture microorganism mixture. Non-limiting example for increased yield include, but not limited to, increased gene expression, protein production and secretion, molecule biosynthesis, proliferation and others. In some embodiments, effective culture conditions include, but are not limited to, effective media, bioreactor, temperature, pH and oxygen conditions that permit for increased production yield. In one embodiment, an effective medium refers to any medium in which a microorganism mixture is cultured to produce a compound of the present invention. In some embodiments, a cell culture medium typically includes an aqueous solution having assimilable carbon, nitrogen and phosphate sources, and appropriate salts, minerals, metals and other nutrients, such as vitamins. In some embodiments, microorganism mixture can be cultured in conventional fermentation bioreactors, shake flasks, test tubes, microtiter dishes and petri plates. In some embodiments, culturing is carried out at a temperature, pH and oxygen content appropriate for a probiotic microorganism, such as a yeast or bacteria. In some embodiments, culturing conditions are within the expertise of one of ordinary skill in the art. A non-limiting example for a process of culturing a microorganism mixture of the present invention comprises culturing the microorganism mixture in milk at 28° C. for about 24 hours.

In one embodiment, the process of culturing comprises culturing the microorganism mixture for a period of 12-16 hours, 14-18 hours, 12-24 hours, 16-24 hours, 18-28 hours, 10-20 hours, 22-36 hours. Each possibility represents a separate embodiment of the invention.

In one embodiment, the process of culturing comprises culturing the microorganism mixture at a temperature of 20-26° C., 24-28° C., 22-34 ° C., 26-34 ° C., 28-38° C., 20-30° C., 32-46° C. Each possibility represents a separate embodiment of the invention.

In some embodiments, the microorganism mixture is cultured in milk. In some embodiments, the microorganism mixture cultured in milk yields a fermented milk product. In some embodiments, the fermented milk product is selected from yogurt, probiotic yogurt, or kefir.

In some embodiments, the fermented milk product comprises a microorganism mixture, Tryptophol derivative, 4-Ethyl-Phenol derivative, or any combination thereof.

In some embodiments, the composition further comprises an acceptable carrier. In some embodiments, the carrier is a pharmaceutical carrier. In some embodiments, the carrier is a nutraceutical carrier.

In some embodiments, the composition is a pharmaceutical composition. In some embodiments, the composition is a nutraceutical composition.

As used herein, the term “carrier” refers to a diluent, adjuvant, excipient, or vehicle with which the active compound is administered. Such carriers can be sterile liquids, such as water-based and oils, including those of petroleum, animal, vegetable or synthetic origin, such as peanut oil, soybean oil, mineral oil, sesame oil and the like, polyethylene glycols, glycerin, propylene glycol or other synthetic solvents.

Water may be used as a carrier such as when the active compound is comprised by a pharmaceutical composition being administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; and agents for the adjustment of tonicity such as sodium chloride or dextrose are also envisioned. The carrier may comprise, in total, from about 0.1% to about 99.99999% by weight of the compositions presented herein.

An embodiment of the invention relates to molecules of the present invention or derivative thereof, presented in unit dosage form and prepared by any of the methods well known in the art of pharmacy. In one embodiment, the unit dosage form is in the form of a tablet, capsule, lozenge, wafer, patch, ampoule, vial or pre-filled syringe.

In addition, in vitro assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems.

In one embodiment, the composition of the present invention is administered in the form of a pharmaceutical composition comprising at least one of the active components of this invention (e.g., Tryptophol derivative, or 4-Ethyl Phenol derivative) together with a pharmaceutically acceptable carrier or diluent. In another embodiment, the composition of the invention can be administered either individually or together in any conventional oral, parenteral or transdermal dosage form.

As used herein, the terms “administering”, “administration”, and like terms refer to any method which, in sound medical practice, delivers a composition containing an active agent to a subject in such a manner as to provide a therapeutic effect.

In some embodiments, the composition comprising the compound of the invention, or any derivative or combination thereof, or the microorganism mixture of the invention, is administered via oral (i.e., enteral), rectal, vaginal, topical, nasal, ophthalmic, transdermal, subcutaneous, intramuscular, intraperitoneal or intravenous routes of administration. The route of administration of the composition will depend on the disease or condition to be treated. Suitable routes of administration include, but are not limited to, parenteral injections, e.g., intradermal, intravenous, intramuscular, intralesional, subcutaneous, intrathecal, and any other mode of injection as known in the art. In addition, it may be desirable to introduce the composition of the invention by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer.

For topical application, the compound of the invention, or any derivative or combination thereof, or the microorganism mixture of the invention, can be combined with a pharmaceutically acceptable carrier so that an effective dosage is delivered, based on the desired activity. The carrier can be in the form of, for example, and not by way of limitation, an ointment, cream, gel, paste, foam, aerosol, suppository, pad or gelled stick.

For oral applications, the composition may be in the form of tablets or capsules, which can contain any of the following ingredients, or compounds of a similar nature a binder such as microcrystalline cellulose, gum tragacanth or gelatin; an excipient such as starch or lactose; a disintegrating agent such as alginic acid, Primogel, or corn starch; a lubricant such as magnesium stearate; or a glidant such as colloidal silicon dioxide. When the dosage unit form is a capsule, it can contain, in addition to materials of the above type, a liquid carrier such as fatty oil. In addition, dosage unit forms can contain various other materials which modify the physical form of the dosage unit, for example, coatings of sugar, shellac, or other enteric agents. The tablets of the invention can further be film coated. In some embodiments, oral application of the composition may be in the form of drinkable liquid. In some embodiments, oral application of the composition may be in the form of an edible product. Non-limiting examples for oral carriers include, but are not limited to milk, yogurt, probiotic yogurt, kefir, fermented milk or others.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes.

The compositions also include incorporation of the active material into or onto particulate preparations of polymeric compounds such as polylactic acid, polglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance.

In one embodiment, the present invention provides combined preparations. In one embodiment, “a combined preparation” defines especially a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. In one embodiment, the combined preparation can be varied, e.g., in order to cope with the needs of a patient subpopulation to be treated or the needs of the single patient which different needs can be due to a particular disease, severity of a disease, age, sex, or body weight as can be readily made by a person skilled in the art.

In one embodiment, it will be appreciated that the molecules of the present invention, or any derivative or combination thereof, or the microorganism mixture of the invention, can be provided to the individual with additional active agents to achieve an improved therapeutic effect as compared to treatment with each agent by itself. In another embodiment, measures (e.g., dosing and selection of the complementary agent) are taken to adverse side effects which are associated with combination therapies.

In one embodiment, depending on the severity and responsiveness of the condition to be treated, dosing can be of a single or a plurality of administrations, with course of treatment lasting from several days to several weeks or until cure is affected or diminution of the disease state is achieved.

In some embodiments, the composition of the preset invention is administered in a therapeutically safe and effective amount. As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the presently described manner. In another embodiment, a therapeutically effective amount of the molecules, or any derivative or combination thereof, is the amount of the mentioned herein molecules necessary for the in vivo measurable expected biological effect. The actual amount administered, and the rate and time-course of administration, will depend on the nature and severity of the condition being treated. Prescription of treatment, e.g., decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington: The Science and Practice of Pharmacy, 21st Ed., Lippincott Williams & Wilkins, Philadelphia, Pa., (2005). In some embodiments, preparation of effective amount or dose can be estimated initially from in vitro assays. In one embodiment, a dose can be formulated in animal models and such information can be used to more accurately determine useful doses in humans.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975) “The Pharmacological Basis of Therapeutics”, Ch. 1 p. 1].

Pharmaceutical compositions containing the molecules disclosed herein, or any derivative or combination thereof, or the microorganism mixture of the present invention, as the active ingredient can be prepared according to conventional pharmaceutical compounding techniques. See, for example, Remington's Pharmaceutical Sciences, 18th Ed., Mack Publishing Co., Easton, Pa. (1990). See also, Remington: The Science and Practice of Pharmacy, 21st Ed., Lippincott Williams & Wilkins, Philadelphia, Pa. (2005).

In one embodiment, compositions including the preparation of the present invention formulated in a compatible pharmaceutical carrier are prepared, placed in an appropriate container, and labeled for treatment of an indicated condition.

In one embodiment, compositions of the present invention are presented in a pack or dispenser device, such as an FDA approved kit, which contains, one or more unit dosages forms containing the active ingredient. In one embodiment, the pack, for example, comprises metal or plastic foil, such as a blister pack. In one embodiment, the pack or dispenser device is accompanied by instructions for administration. In one embodiment, the pack or dispenser is accommodated by a notice associated with the container in a form prescribed by a governmental agency regulating the manufacture, use or sale of pharmaceuticals, which notice is reflective of approval by the agency of the form of the compositions or human or veterinary administration. Such notice, in one embodiment, is labeling approved by the U.S. Food and Drug Administration for prescription drugs or of an approved product insert.

### General

As used herein the term “about” refers to ±10%.

The terms “comprises”, “comprising”, “includes”, “including”, “having” and their conjugates mean “including but not limited to”.

The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure. As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

Reference is now made to the following examples, which together with the above descriptions illustrate some embodiments of the invention in a non-limiting fashion.

Generally, the nomenclature used herein, and the laboratory procedures utilized in the present invention include molecular, biochemical, microbiological and recombinant DNA techniques. Materials and Methods

To explore the effect of the molecules as described herein on the gut microbiome, the inventors used two different models examined in mice. First, the inventors used the model of dextran sodium sulfate (DSS) to induced acute intestinal inflammation as occurs in Crohn's disease and/or colitis diseases, which resemble human inflammatory bowel disease (IBD). All mice groups were treated with DSS dissolved in their drinking water for a period of 7 days. The molecules-treated group received a mixture of Tyrosol acetate and Tryptophol acetate, that was dissolved in water and delivered using oral gavage directly to the gastrointestinal (GI) tract at the time of DSS treatment. The inventors performed 3 different experiments, wherein various concentrations dose of the molecules, e.g., Tyrosol acetate and Tryptophol acetate, were used, namely, 25 μM each, 50 μM each, or 75 μM each. Each group included 6 mice.

To explore the bacterial abundance and diversity in the gut microbiome of the mice following the molecules treatment, the inventors examined the intestinal bacterial population using DNA sequencing of fecal samples that were collected from all mice participating in the experiment, both pre-therapy (1^{st} day) and at the end point day.

Next, the inventors used the model of lipopolysaccharides (LPS) stimulation, known to induce a cytokine storm in mice. The inventors preformed the experiments with two groups. Both groups were stimulated by injection of LPS at a dose sufficient to induce a cytokine storm (30 mg/kg). The group of mice that was also treated with the molecules, received the molecules mixture dissolved in water with concertation of 75 μM using oral gavage directly to the GI tract at the time of LPS treatment. Then, the inventors examined the effect of molecules on intestinal bacterial population using DNA sequencing of fecal samples that were collected from all mice participating in the experiment, at time 0, and 6 h and 24 h after LPS administration.

### Example 1

### Alteration of Gut Microbial Populations

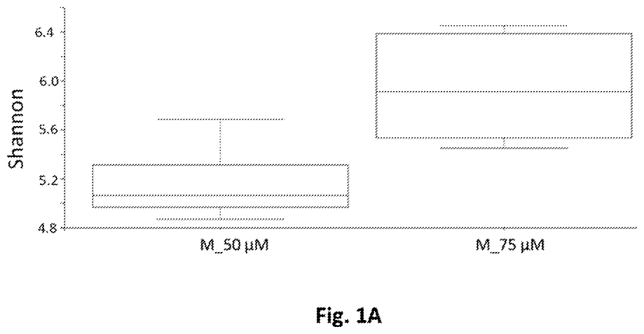

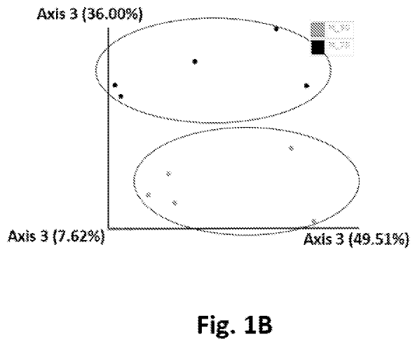

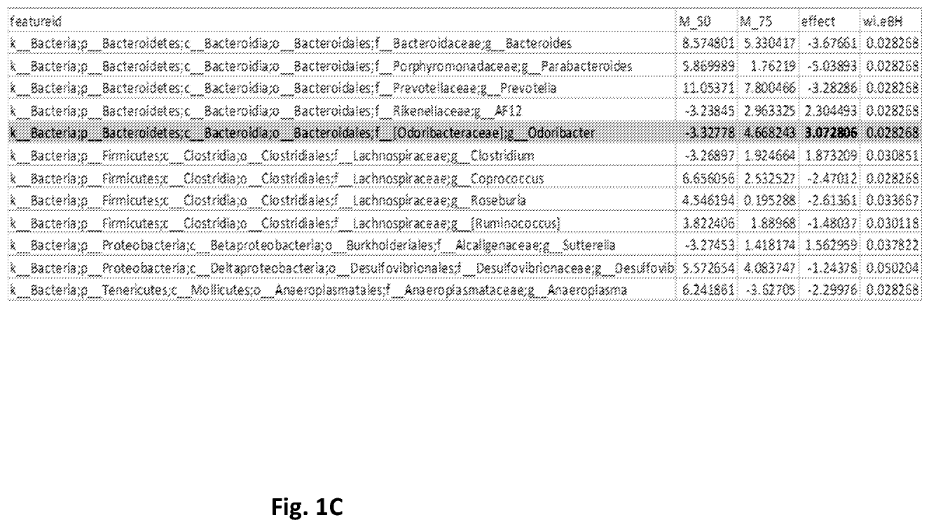

Shannon's diversity index (FIG. 1A) and beta diversity indices (FIG. 1B) showed statistically significant dissimilarities between mice groups that were treated with different doses. Additionally, ANOVA-like Differential Gene Expression showed an increase in the presence of Odoribacteraceae—a family in the order of Bacteroidetes, exclusively in the population of mice were treated with the molecules, each at a concentration of 75 μM (FIG. 1C).

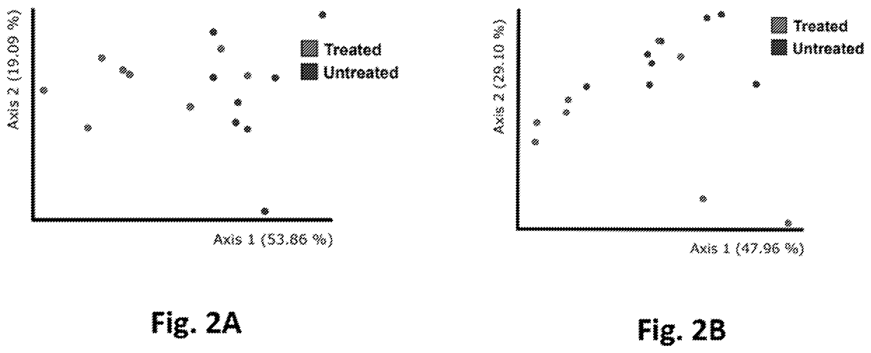

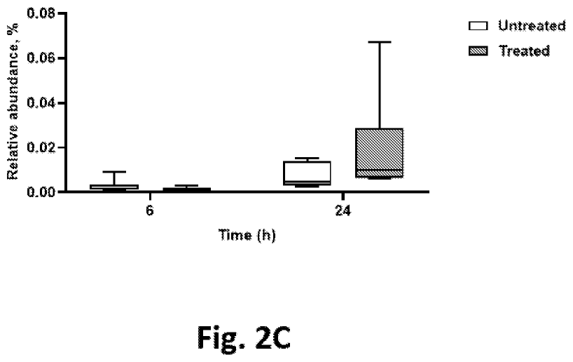

Due to the above observations that administration of tryptophol acetate and tyrosol acetate orally gave rise to the pronounced anti-inflammatory effects, the inventors further assessed the effects of the molecules on the gut microbiota. The relationship between severe inflammation and microbial dysbiosis has been extensively studied in recent years. The gut microbiota has been shown to enhance host immunity to pathogens, and dysbiosis has been linked to increased susceptibility of severe inflammation. Accordingly, the inventors characterized the gut microbial compositions of mice prior and after LPS injection, and with and without treatment with the mixture of tryptophol acetate and tyrosol acetate. Indeed, oral administration of the molecules had a significant impact on the community composition, manifested by an increase in the abundance of bacteria with anti-inflammatory properties (Tables 1-2). The inventors found differences in beta diversity (i.e., between sample diversities; FIGS. 2A, 2B) between treated and untreated mice 6 and 24 hours after LPS injection (PERMANOVA, P=0.001 and P=0.038, respectively). This important observation indicates that dysbiosis (shift in the microbiome) at the community level was already observed 6 hours after LPS administration. Notably, the predominant change recorded was the increase in the genus Bacteroides in mice that were treated with the molecules 24 hours after LPS administration (Tables 1-2 and FIG. 2C).

As can be seen in the results presented in Tables 1 and 2, this taxon was also abundant in the microbiome of healthy mice. Those results are important as Bacteroidetes has been linked to various immune-protective and anti-pathogenic activities associated with microbiome modifications.

TABLE 1
Results after 6 hours from LPS administration.
ANCOM results

| | | Treated | |
|---|---|---|---|
| | | (Molecules + | Untreated |
| OUTs | Healthy | LPS) | (LPS only) |
| g_Blautia | High | Low | Low |
| g_Bacteriodes | High | Low | Low |
| g_Clostridium | High | Low | Low |
| g_Sutterella | High | Low | Low |
| g_Ruminococcus | Low | High | High |
| g_Parabacteriodes | High | Low | Low |

TABLE 2
Results after 24 hours from LPS administration.
ANCOM results

| OTUs | Healthy | Treated | Untreated |
|---|---|---|---|
| g_Blautia | High | Low | Low |
| g_Bacteriodes | High | High | Low |
| g_Clostridium | High | Low | Low |
| g_Oscillospira | Low | High | High |
| g_Ruminococcus | Low | High | High |

Recent studies have reported on the anti-inflammatory properties of Bacteroides. The mechanisms of action proposed for these anti-inflammatory activities include inhibition of pathogen colonization and increased mucosal barrier by modifying goblet cells and mucin glycosylation. The higher abundance of Bacteroides upon treating the LPS-injected mice with tryptophol acetate and tyrosol acetate may thus account to “cross-talk” between the molecules and host microbiota, promoting intestinal homeostasis. Thereby, consumption of tryptophol acetate and tyrosol acetate in combination with antibiotics may present a new approach for reducing the harmful side effects of antibiotics on host gut microbiome.

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for modulating abundance, diversity, or both, of a microbial population in a subject in need thereof, comprising administering to said subject a therapeutically effective amount of a composition comprising a Tryptophol derivative, a 4-Ethyl-Phenol derivative, or a combination thereof, thereby modulating abundance, diversity, or both, of a microbial population in the subject.

2. The method of claim 1, wherein said modulating comprises increasing or decreasing.

3. The method of claim 2, wherein said increasing or decreasing is by at least 5% compared to a control.

4. The method of claim 1 to 3, wherein said modulating comprises increasing the abundance, diversity, or both, of bacteria belonging to the phylum of Bacteriodetes.

5. (canceled)

6. The method of claim 1 to 5, wherein said modulating comprises reducing the abundance, diversity, or both, of bacteria belonging to a family selected from the group consisting of Vibrionaceae, Enterobacteriaceae, Staphylococcaceae, Pseudomonadaceae, Helicobacteraceae, and any combination thereof, bacteria selected from the group consisting of Vibrio cholerae, salmonella enterica, staphylococcus aureus, Pseudomonas aeruginosa, Helicobacter pylori, and any combination thereof, or any combination thereof.

7. (canceled)

8. The method of claim 1, wherein said composition comprises any one of said Tryptophol derivative and said 4-Ethyl-Phenol derivative in a concentration ranging from 1 μM to 100 μM.

9. The method of claim 1, wherein said Tryptophol derivative is Tryptophol acetate, wherein said 4-Ethyl-Phenol derivative is Tyrosol acetate, or combination thereof.

10. (canceled)

11. The method of claim 1, wherein said microbial population comprises a skin microbial population, a gut microbial population, a vaginal microbial population, or any combination thereof, of said subject.

12. The method of claim 1, wherein said modulating abundance, diversity, or both, of said microbial population in said subject comprises modulating abundance, diversity, or both, of said microbial population in skin, digestive system, oral cavity, respiratory system, a body topical surface, gut, vagina, or any combination thereof, of said subject.

13. The method of claim 1, wherein said subject is afflicted with an inflammatory disease.

14. The method of claim 1, wherein said subject is afflicted with an inflammatory bowel disease (IBD), comprising any one of Crohn's disease and ulcerative colitis.

15. (canceled)

16. The method of claim 1, wherein said composition is a pharmaceutical composition or a nutraceutical composition.

17. A method for treating a subject in need of microbial population modulation, comprising administering a composition comprising a Tryptophol derivative, a 4-Ethyl-Phenol derivative, or a combination thereof, a microorganism mixture and an acceptable carrier.

18. (canceled)

19. (canceled)

20. (canceled)

21. (canceled)

22. (canceled)

23. The method of claim 17, wherein said microorganism mixture comprises Kluyveromyces marxianus and at least one probiotic microorganism, and wherein said microorganism mixture comprises at least 3% K. marxianus.

24. The method of claim 23, wherein at least one probiotic microorganism is a probiotic bacterium.

25. The method of claim 24, wherein said probiotic bacterium is selected form the group consisting of Lactobacillus, Propionibacterium, Lactococcus, and Leuconostoc.

26. The method of claim 17, wherein said microorganism mixture is suspended in a medium.

27. (canceled)

28. The method of claim 17, wherein said microorganism mixture is kefir.

29. The method of claim 17, wherein said Tryptophol derivative and said 4-Ethyl-Phenol derivative are produced by K. marxianus.

30. The method of claim 17, being a pharmaceutical composition or nutraceutical composition.
